(19) United States
(12) Patent Application Publication
(10) Pub. No.: US 2023/0204742 A1
(43) Pub. Date: Jun. 29, 2023

(54) LOW-COST, HIGH-PERFORMANCE ULTRASOUND IMAGING PROBE

(71) Applicant: Exo Imaging, Inc., Santa Clara, CA (US)

(72) Inventors: Yusuf HAQUE, Woodside, CA (US); Janusz BRYZEK, Santa Clara, CA (US)

(21) Appl. No.: 18/091,288

(22) Filed: Dec. 29, 2022

Related U.S. Application Data
(60) Provisional application No. 63/294,717, filed on Dec. 29, 2021.

(51) Int. Cl. G01S 7/52; A61B 8/00; A61B 8/08; H04N 19/10

(52) U.S. Cl. CPC G01S 7/52034; G01S 7/52085; A61B 8/56; A61B 8/4488; A61B 8/5207; A61B 8/483; H04N 19/10

(57) ABSTRACT

An ultrasonic imaging system can include an ultrasound imaging probe, a computing device, and a link for communicatively coupling the computing device and the ultrasound imaging probe. The probe can include an ultrasonic transducer and preprocessing circuitry. The ultrasonic transducer can produce an electrical signal from an ultrasonic pressure wave and have a transducer element. The preprocessing circuitry can be electrically coupled to the ultrasonic transducer and have a signal converter and a signal integrator. The signal converter can condition a signal from the transducer element and convert the signal to a digital signal. The signal integrator can combine the digital signal into a transmission signal with at least a 10 Gigabit per second data rate. The signal can then be transmitted for processing by the computing device.

### CROSS-REFERENCE TO RELATED APPLICATIONS

The present application is a nonprovisional of U.S. Provisional Patent No. 63/294,717, filed on Dec. 29, 2021, the entirety of which is incorporated herein by reference.

### BACKGROUND

Ultrasound technology enables non-invasive imaging of tissue and can be useful in many non-medical contexts as well, such as in industrial manufacturing. Handheld ultrasound imaging devices have been developed to enhance the portability of ultrasound technologies. Such handheld ultrasound imaging devices may contain various circuit components for generating, processing, and digitizing ultrasound signals within a small, handheld package.

### SUMMARY

Some embodiments disclosed herein provide a handheld ultrasound imaging device that can provide advanced capabilities, such as the reconstruction of three-dimensional images. In accordance with at least some embodiments disclosed herein is the realization that three-dimensional ultrasonic imaging, high-resolution imaging, and other advanced ultrasound capabilities may generate large amounts of raw data for conversion into ultrasound images. Further, some embodiments also relate to the realization that electronics used to convert the ultrasound signals into high-resolution images, if included in the ultrasound imaging device, may draw substantial power, generate substantial heat, and may be expensive. Thus, some embodiments disclosed herein address a need for an improved ultrasound device and system that retains advanced image processing capabilities while minimizing drawbacks.

For example, in accordance with at least some embodiments disclosed herein is the realization that emerging high-speed communication technologies, such as Universal Serial Bus 4 (USB4), significantly increase the amount of data that a device can transmit to another device, providing data transmissions of up to 40 gigabits per second (Gbps) or more. Such high-speed technologies can be incorporated into some embodiments of the ultrasound imaging device and/or system disclosed herein in order to allow the ultrasound imaging device to transmit increased amounts of ultrasound data to third party computing and/or display devices.

Accordingly, some embodiments of the present disclosure provide an advantage over existing technologies not just through the use of high-speed data transfer technology, but in the offloading of processing demands to third-party devices, thus enabling other features and functions to be achieved using available space, computing, and power of the ultrasound imaging device that would have otherwise been required for processing data. This advantageously allows for various innovations and improvements to the ultrasound imaging devices themselves, as discussed herein.

For example, some embodiments of the ultrasound imaging devices disclosed herein can consume less power, be more compact, be more lightweight, and/or be made more inexpensively in order to allow wider access to such technology, whether to average consumers or to enable such devices to be ubiquitously incorporated into doctor offices and used in routine visits. Thus, these improvements and benefits can enable the public and medical professionals the ability to utilize better data and develop new practices that can improve healthcare and patient outcomes.

Furthermore, some embodiments also provide an imaging system that can incorporate the use of third-party computing devices, which may be remote or less-size-constrained than the presently disclosed the imaging devices. Accordingly, such systems can be more capable, flexible, and adaptable than existing technologies and enable a clinician/user to pair the imaging device with at least one of a variety of third-party, conventional processing and/or display devices, whether only one or multiple devices, in order to perform computation-intensive tasks to produce higher-resolution ultrasound images and provide feedback and analytics, as appropriate, in accordance with some embodiments. Indeed, such third-party computing device(s) may be used with some embodiments of the system disclosed herein to enable the user to benefit from increased amounts of ultrasound data, for example, in machine learning or deep learning.

Accordingly, utilizing various surprising and unexpected benefits of some embodiments disclosed herein, a user can obtain highly customizable feedback or instruction during use of the device. Further, some embodiments enable the device and system to customize data transmission and related functions based on a selected procedure type. As noted herein, such advantageous features and functions of some embodiments disclosed herein are feasible by creatively leveraging communication technologies and the power of modern smartphones and smart tablets. Thus, some embodiments of the present disclosure provide substantial innovations and improvements to ultrasound imaging probe technology.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an ultrasound imaging system, in accordance with some embodiments.

FIG. 2 illustrates a schematic diagram of an imager, in accordance with some embodiments.

FIG. 3 illustrates a process for producing a data transmission from an ultrasound signal, in accordance with some embodiments.

FIG. 4 illustrates a process for creating a high-resolution image from a serial bit stream, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Overview

Disclosed is an ultrasonic imaging system that leverages high-throughput connections (e.g., USB4, PCI-E, PXIE) to provide a low-cost, high-resolution ultrasound image. The disclosed imaging system includes an ultrasound imaging probe, a computing device, and a communication link between the two. Because postprocessing operations may be offloaded to the computing device, the imager's weight, size, power and cost may be reduced.

The ultrasound imaging probe transmits (in a transmit mode) and receives ultrasonic pressure waves into and from a medium (in a receive mode), performs preprocessing of received ultrasonic signals, and provides the received signals to a computing device. The ultrasound imaging probe may include one or more transducer elements. The transducer elements may be piezoelectric micromachined transducers (pMUTs) (e.g., transducers using piezoelectric elements comprising aluminum nitride (AlN) or lead zirconate titanate (PZT)), or capacitive micromachined transducers (cMUTs). The transducer elements may be organized in an array.

Additionally, the ultrasound imaging probe includes preprocessing electronics, which may be integrated electronic circuits. The preprocessing electronics may include one or more digital signal processors. The preprocessing electronics may include one or more analog-to-digital converters (ADCs) for digitizing signals received by the transducer elements. The preprocessing electronics may include a signal converter, for conditioning signals from the ultrasonic transducer and converting the signals to digital signals. Signal conditioning may include processes, such as filtering, amplification, attenuation, and electrical isolation, that are implemented prior to converting the signal into a digital signal for more accurate conversion.

The preprocessing electronics may include microbeamforming electronics for providing a large number of ultrasonic signals received from transducer elements to a smaller number of transducer channels. Using microbeamforming, transducer element signals may be divided into subsets, may have individual delays applied to the signals in the subsets, and may then be summed before being provided to the transducer element channels. In this manner, for example, 100,000 signals from 100,000 transducer elements may be provided to 1024 channels, while persisting the information from each transducer signal such that a particular signal may be reconstructed by the third-party computing device. The preprocessing electronics may also include additionally circuitry, such as a signal integrator for combining the digital signals into a high-speed transmission signal (e.g., a 10 Gbps signal, a 20 Gbps signal, a 40 Gbps signal) for transmitting the bit stream (e.g., externally) and for receiving one or more beamforming instructions. In some embodiments, the transmission signal may be a serial signal, such as a single serial signal or a multiple serial signal.

For example, the high-speed transmission interface may be a Universal Serial Bus 4 (USB4) interface, USB3, Peripheral Component Interconnect Express (PCI-E), or PCI eXtensions for Instrumentation Express (PXIE). The high-speed transmission interface may enable serial transmission of data at up to 40 Gbps. The high-speed transmission interface may comprise a converter than can be a serializer, which may convert parallel signals collected from multiple transducers into serial bit streams. The serial bit streams may convey the amplitude or phase information from the received ultrasonic signals.

The computing device provides beamforming instructions for transmission and/or receipt of ultrasound signals and receives the serial bit stream from the ultrasound imaging probe. The beamforming instructions may program time delays into individual transducer elements. When the computing device receives the serial bit stream, the computing device creates an image reconstruction. To create a high-resolution ultrasound image, the computing device may implement one or more post-processing algorithms on the serial bit stream.

The communication link couples the computing device and the ultrasound imaging probe. The communication link may be a physical cable (e.g., a USB4 cable, a USB3 cable, a PCI-E cable, or a PXIE cable) or the communication link may be a wireless connection, such as a cellular (e.g., 5G) or Bluetooth connection. The communication link may transmit data unidirectionally or bidirectionally.

The communication link may provide beamforming instructions from the computing device to the ultrasound imaging probe. The communication link may provide a serialized, digitized signal from the ultrasound imaging probe to the computing device.

### Imaging System

In an aspect, an ultrasonic imaging system is disclosed. The ultrasonic imaging system may comprise an ultrasound imaging probe. The ultrasound imaging probe may comprise at least one ultrasonic transducer element (also referred to herein as an ultrasound transducer element) and circuitry electrically coupled to the ultrasonic transducer element or transducer elements. The ultrasonic imaging system may also comprise a computing device (interchangeably referred to as a “third-party computing device”) and a link for communicatively coupling the computing device and the ultrasound imaging probe.

The ultrasonic transducer element may be a device that converts an ultrasonic pressure wave into an electrical signal (in a receive mode) or an electrical signal into an ultrasonic pressure wave (in a transmit mode). The pressure wave may be in the form of a pulse. An ultrasonic transducer element may be a pMUT transducer element or a cMUT transducer element. In some embodiments, there may be more than about 1, more than about 10, more than about 50, more than about 100, more than about 500, more than about 1000, more than about 2000, more than about 5000, more than about 10,000, more than about 20,000, or more than about 50,000 transducer elements. In some embodiments, there may be fewer than about 10, fewer than about 50, fewer than about 100, fewer than about 500, fewer than about 1,000, fewer than about 5,000, fewer than about 10,000, fewer than about 20,000, fewer than about 50,000, or fewer than about 100,000 transducer elements. In some embodiments, there may be between 1 and 10, between 10 and 50, between 50 and 100, between 100 and 1,000, between 1,000 and 5,000, between 5,000 and 10,000, between 10,000 and 50,000, or between 50,000 and 100,000 transducer elements. The transducer elements may be disposed in an array (e.g., a rectangular array, a square array, a circular array, a hexagonal array, or an array of another shape). For arrays of square or rectangular shape, the pMUT elements may be indexed by row and column to enable control of individual delay elements.

The circuitry may preprocess a received ultrasound signal. The circuitry may include a low noise amplifier (LNA), one or more analog-to-digital converters (ADCs), a signal processor, and a data compressor. In other embodiments, the preprocessing circuitry may include additional signal or data processing components.

The circuitry may be provided using one or more application-specific integrated circuits (ASICs). For example, preprocessing may be provided by a single ASIC including an LNA, analog front-end circuitry, and a data compressor. In other embodiments, the ASIC may provide further signal processing capabilities. In some embodiments, an ASIC may have more than 1, more than 4, more than 8, more than 16, more than 32, more than 64, more than 128, more than 256, or more than 512 channels. In some embodiments, an ASIC may have fewer than 16, fewer than 32, fewer than 64, fewer than 128, fewer than 256, fewer than 512, fewer than 1024, or fewer than 2048 channels. The circuitry may have two digital signal processors (DSPs). For example, outputs from a plurality of transducers may be microbeamformed by a first DSP, and then further processed by a second DSP.

The LNA may amplify an electrical signal produced by a transducer element without significantly reducing its signal-to-noise ratio. Thus, a transducer signal may be amplified by the LNA prior to any further preprocessing.

The ADCs may provide resolution of greater than 1 bit, greater than 4 bits, greater than 8 bits, or greater than 12 bits, greater than 14 bits, greater than 16 bits, or greater than 20 bits. The ADCs may provide resolution of fewer than 4 bits, fewer than 8 bits, fewer than 12 bits, fewer than 14 bits, fewer than 16 bits, or fewer than 20 bits.

The circuitry may produce high-resolution digital data from the received electrical signals. The data may be a parallel data stream. The data may have a data rate of more than 10 Gbps, more than 20 Gbps, more than 30 Gbps, more than 40 Gbps, more than 50 Gbps, more than 60 Gbps, or more than 70 Gbps. The data may have a data rate of less than 30 Gbps, less than 40 Gbps, less than 50 Gbps, less than 60 Gbps, less than 70 Gbps, or less than 80 Gbps.

The circuitry may comprise data compression circuitry to compress a digitized signal. The data compression circuitry may implement lossless or lossy data compression. The data compression circuitry may implement compression algorithms such as entropy coding, Huffman coding, Lempel-Ziv-Welch (LZW) algorithm, block floating point encoding, or another compression technique.

The ultrasound imaging probe may comprise a high-speed transmission converter. In some embodiments, the high-speed transmission converter may be a serial converter and may be configured to produce a serial bit stream with a speed of at least 20 Gbps, at least 30 Gbps, at least 40 Gbps, at least 50 Gbps, at least 60 Gbps, or at least 70 Gbps. By producing a high-speed serial bit stream, the serial converter enables the digitized ultrasound signal to be transported to the computing device for postprocessing.

The ultrasound imaging probe may also include a high-speed transmission interface for transmitting the bit stream (e.g., externally) and for receiving beamforming instructions from the computing device. The high-speed transmission interface may comprise a USB4 interface, a USB3 interface, a PCI-E interface, or a PXIE interface.

The ultrasonic imaging system may also include a computing device. The computing device may receive the bit stream and may implement one or more signal processing operations on the bit stream to produce one or more ultrasound images.

The computing device may also provide beamforming instructions to the ultrasound imaging probe. The beamforming instructions may implement time delays on one or more of the transducer elements of the ultrasound imaging probe. The beamforming instructions may be transmitted over the communication link from the computing device to the ultrasound imaging probe. The circuitry of the ultrasound imaging probe may convert the instructions to analog electrical signals which are provided to the transducer elements. The transducer elements may convert these signals to pressure waves, which may be emitted as pulses from the transducer elements according to delay information conveyed in the signals.

The computing device may be a desktop computer, laptop computer, smartphone (e.g., an iPhone or Android phone), personal digital assistant (PDA), tablet computer (e.g., an APPLE® iPad Pro, an APPLE® iPad Air, a MICROSOFT® Surface, or a SAMSUNG® Galaxy Tab), mainframe computer, supercomputer, or other type of computer, or cloud computing device. The computing device may implement a MICROSOFT® Windows™, APPLE® Macintosh™, Linux, Unix, GOOGLE® CHROME Operating System, Android operating system, iOS operating system, or another operating system. In some embodiments, postprocessing tasks may be implemented on multiple computers. The computing devices may transmit ultrasound image data to one another over wired or wireless networks. The computing devices may use cloud computing to upload and download ultrasound image data.

The computing devices may produce two-dimensional (2D), three-dimensional (3D), or four-dimensional (4D) images. The computing devices may produce A-mode, B-mode, B-flow, C-mode, M-mode, Doppler-mode, pulse inversion mode, and/or harmonic mode images. The computing devices may produce 3D or 4D images with frame rates of greater than 10 frames per second (FPS), greater than 20 FPS, greater than 30 FPS, greater than 50 FPS, greater than 100 FPS, greater than 200 FPS, greater than 300 FPS, greater than 400 FPS, greater than 500 FPS, greater than 600 FPS, greater than 700 FPS, greater than 800 FPS, or greater than 900 FPS. The computing devices may produce 3D or 4D images with frame rates of less than 20 frames per second, less than 30 FPS, less than 50 FPS, less than 100 FPS, less than 200 FPS, less than 300 FPS, less than 400 FPS, less than 500 FPS, less than 600 FPS, less than 700 FPS, less than 800 FPS, less than 900 FPS, or less than 1000 FPS.

The ultrasonic imaging system may also include a link for communicatively coupling the computing device with the ultrasound imaging probe. The link may be a high-speed serial wired link. The link may be a USB4 link, a USB3 link, a PCI-E link, or a PXIE link. In addition to providing data to the computing device and/or to the ultrasound imaging probe, the link may also provide power to the computing device and/or the ultrasound imaging probe.

### DESCRIPTION OF THE FIGURES

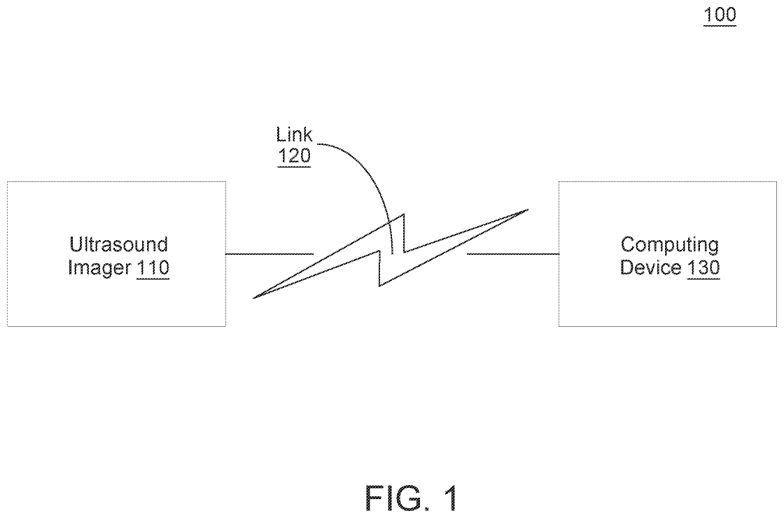

FIG. 1 illustrates an ultrasound imaging system 100, in accordance with some embodiments. The ultrasound imaging system includes an ultrasound imaging probe 110, a link 120, and a computing device 130.

The ultrasound imaging probe 110 transmits pressure waves into tissue of a subject (e.g., organ tissue of a human subject) in a transmit mode or process and receives pressure waves reflected from the tissue. The ultrasound imaging probe may also include circuitry that may send signals to and receive signals from the computing device 130 over the link 120. In embodiments, tissue of the subject may reflect a portion of transmitted pressure waves to the imager 110 and the imager 110 may capture the reflected pressure waves and generate electrical signals in a receive mode process. The imager 110 may communicate electrical signals to the computing device 130 and the computing device 130 may display images of the tissue on a display or screen.

In some embodiments, the imager 110 may be used to capture images from nonhuman subjects (e.g., mammalian or non-mammalian animals). Images produced from imager data may determine velocity of blood flow in arteries and veins as in Doppler mode imaging and also measure tissue stiffness. In some embodiments, a pressure wave may be an acoustic wave that may travel through a subject body and be reflected by subject body tissue, arteries, or veins.

In some embodiments, the imager 110 may be a portable device and communicate signals through the link 120. As noted above, the link 120 may include a wired connection, such as a high-speed serial interface (e.g., USB4, USB3, PCI-E, or PXIE). The link 120 may also include a wireless connection, such as a cellular (e.g., 5G) or Bluetooth interface. Due to the potential increase in bandwidth via the link 120, in some embodiments, the imager 110 may transmit significant amounts of imaging data at high rates (e.g., at least 20 Gbps, at least 30 Gbps, at least 40 Gbps, at least 50 Gbps, at least 60 Gbps, or at least 70 Gbps) to the computing device 130 via the link 120.

With increased amounts of imaging data available to the computing device 130, the computing device 130 may therefore perform functions requiring significant amounts of imaging data, such as deep learning (e.g., beamforming using deep learning). Thus, in some embodiments, the computing device 130 can include a deep learning or a machine learning module. For example, the computing device 130 may include a deep learning module trained with imaging data to produce beamforming instructions when provided with imaging data. In some embodiments, the computing device 130 may be a mobile device, such as a smartphone or tablet, or a stationary computing device.

In some embodiments, the link 120 may be used to transmit power from the computing device 130 to the imager 110. For example, the imager 110 may receive some or all of the power it requires from the computing device 130. This may allow the imager 110 to run on a smaller battery than it otherwise would if it did not receive power from the computing device 130. Or the imager 110 may not require a battery at all if the computing device 130 provides enough power to the imager 110. As discussed herein, the high-speed nature of the link 120 may enable the imager 110 to offload data processing to the computing device 130, thus reducing the power requirements of the imager 110 and increasing the possibility that the computing device 130 can supply all of the power required by the imager 110. In any event, transmitting power from the computing device 130 to the imager 110 via the link 120 may reduce the overall cost, weight, or size of the imager 110 (e.g., because the imager 110 does not require an internal battery).

In embodiments, more than one imager may be used to develop an image of the target organ. For instance, the first imager may send the pressure waves toward the target organ while the second imager may receive the pressure waves reflected from the target organ and develop electrical charges in response to the received waves. In ultrasound tomography, transmitters may be located on one side of the body of the imager, and receivers on the other side of the body of the imager.

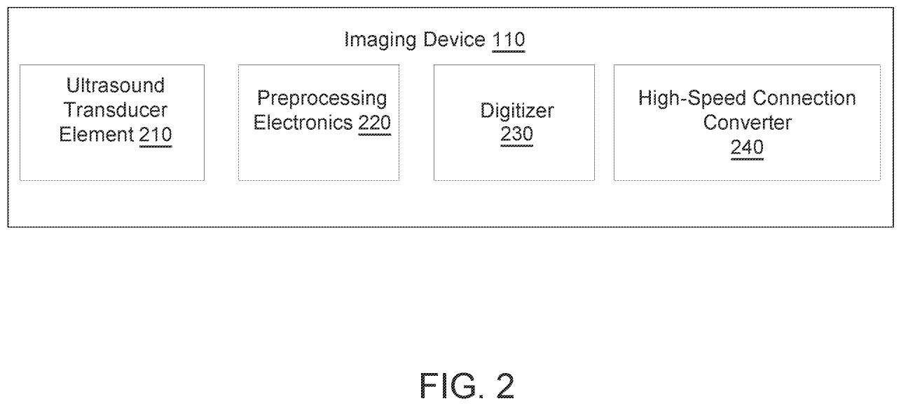

FIG. 2 illustrates a schematic diagram of the imager 110. The imager 110 includes one or more ultrasound transducers 210, preprocessing electronics 220, a digitizer 230, and a high-speed connection converter 240.

An ultrasound transducer produces an electrical signal when impacted by a reflected ultrasound wave and generates an ultrasound pressure wave in response to an electrical signal. The ultrasound transducer may be a micromachined ultrasonic transducer (MUT) and may be piezoelectric (pMUT) or capacitive (cMUT). Generally, a pMUT may include a membrane layer suspended from a substrate, a piezoelectric layer deposited on all or a portion of the membrane, and two electrodes enabling actuation of the piezoelectric layer. In some embodiments, pMUTs may include additional piezoelectric layers sandwiched between additional electrodes. Generally, a cMUT may comprise a silicon substrate with a membrane suspended from the substrate and shallow cavity formed under the membrane. When an alternating voltage is applied across the membrane and substrate, it produces a vibration yielding a pressure wave in transmit mode. When a pressure wave is incident on the membrane in receive mode, the vibration of the membrane causes a change in capacitance, which may be measured as an electrical signal.

Pre-processing electronics 220 implement one or more preprocessing algorithms on the received ultrasonic signals. Preprocessing may mitigate degradation relating to physical properties of the received ultrasound signals (e.g., bandwidth, nonlinear propagation, attenuation, or absorption). Preprocessing algorithms may be implemented on analog signals received from the transducer or on signals that have been digitized by one or more ADCs. Preprocessing algorithms may provide time gain compensation, selective enhancement, log compression, fill-in interpolation, edge enhancement, image updating, data compression, frequency filtering, or multichannel signal aggregation.

Digitizer 230 may use one or more analog-to-digital converters (ADCs) to convert the ultrasonic signal into a digital signal. An ADC may typically have resolution of between 8 bits and 16 bits. The digitizer may implement one ADC per transducer element or may have individual ADCs convert signals from multiple transducer elements.

The high-speed connection converter 240 converts the pre-processed ultrasound data to be transmitted over a high-speed transmission interface. The high-speed connection converter may comprise a serializer, which may convert the pre-processed ultrasound signal from multiple channels in a serial bit stream. For example, the data from each ultrasound transducer may be converted using an ADC to produce a digital ultrasound waveform with an amplitude and a phase component. At this stage, the received waveforms from the ultrasound transducers may be parallel data. A serializer operating at a higher frequency than those of the parallel data channels may control placement of individual bits of data from each of the channels into a serial bit stream.

The high-speed connection may include a single or one serial link, as with a USB4 connection. The USB4 connection may be able to provide speeds of up to 40 Gbps. The high-speed connection may also include multiple serial links, as with a PCI-E connection or a PXIE connection.

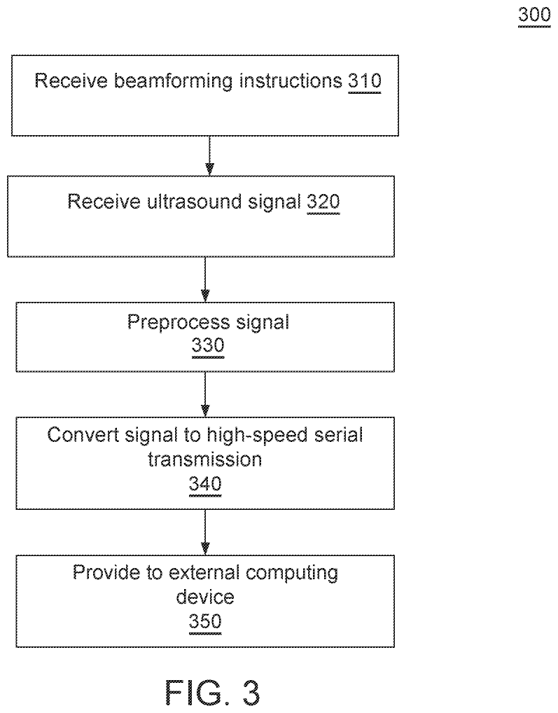

FIG. 3 illustrates a process 300 for producing a high-speed data transmission from one or more ultrasound signals. The high-speed data transmission may be produced using an ultrasound imaging probe.

In a first operation 310, the ultrasound imaging probe can receive beamforming instructions from the computing device. In some embodiments that incorporate beamforming components and function, the device can advantageously have a comparatively lower amount of data to being sent to the computing device (when compared to traditional technology). Such embodiments can also advantageously use a higher number of transducers in the ultrasound imaging probe than traditional technologies, resulting in improved data collection and resolution capabilities.

In some embodiments, to reduce the amount of data, signals from adjacent and/or nearby transducers can be combined. However, in accordance with some embodiments is the realization that loss or abstraction of data, including spatial information, can result from the combination of data of adjacent transducers into one signal. Accordingly, beamforming instructions may advantageously apply a delay or a weight to signals from adjacent or nearby transducers prior to combining the signals—thus creating a beam (or a “microbeam”) that preserves spatial identity while also reducing data rate.

In some embodiments, the computing device may perform beamforming on raw transducer data. The computing device can send the beamforming instructions over a high-speed link, such as a single or multiple serial link. Beamforming instructions are required for transmitting and receiving operations. For transmission, the beamforming instructions may comprise an amplitude, a number of pulses, a delay, a period, a frequency, or a phase for a pulse for a particular transducer. The beamforming instructions may be converted into an electrical signal by one or more circuits within the ultrasound imaging probe. The ultrasound imaging probe may then provide the electrical signal to one or more transducer elements, which produce a pressure wave from the electrical signal and directs it into tissue of a subject. The tissue reflects the pressure wave back to the ultrasound imaging probe.

In a second operation 320, the ultrasound imaging probe receives the reflected pressure wave. In some embodiments, multiple transducer elements within the imager may receive portions of the reflected pressure wave. The transducer elements then convert the portions of reflected pressure wave into electrical signals.

In a third operation 330, the ultrasound imaging probe preprocesses the electrical signals produced by the transducer elements. In some embodiments, the ultrasound imaging probe may carry out received beamforming instructions. At a high level, this may include summing signals from various ultrasound transducers (e.g., without delaying the signals), weighing the signals, or windowing the signals with interpolations between sample events.

As another example, the ultrasound imaging probe may use a low noise amplifier (LNA) to amplify the electrical signals without adding significant amounts of noise. In some embodiments, the ultrasound imaging probe includes a respective LNA for each transducer element. In some embodiments, an imaging device with 1,000 ultrasound transducer elements might include 1,000 LNAs, where each LNA is dedicated to amplifying a respective ultrasonic signal from a respective ultrasound transducer element.

As yet another example, the ultrasound imaging probe may digitize the electrical signals (e.g., after amplifying the signals) using one or more analog-to-digital converters (ADCs). In some embodiments, the ultrasound imaging probe includes a respective ADC for each transducer element. In some embodiments, an imaging device with 1,000 ultrasound transducer elements might include 1,000 ADCs, where each ADC is dedicated to converting a respective ultrasonic signal from a respective ultrasound transducer element into a digital signal. However, in some embodiments, there may be many more transducer elements than ADC channels. In such embodiments, a group of transducer elements may be configured to provide signals to a single channel using microbeamforming. In some embodiments, there may be up to 100,000 transducer elements and/or up to 1024 ADC channels. The ADCs may have resolution between 8 and 16 bits for quantizing the electrical signals.

At times, the amount of data produced by the ultrasound imaging probe may exceed the amount of data that can be sent to the computing device. In some embodiments, an ultrasound imaging probe collecting 3D or 4D imaging data may collect imaging data at a rate higher than it can send the imaging data to the computing device. Further, an ultrasound imaging probe with thousands (e.g., 10,000+, 100,000+) of ultrasound transducer elements may collect imaging data at a rate higher than it can send the imaging data to the computing device. Accordingly, the ultrasound probe may need to compress the data, manipulate the data, and/or store the data in order to account for bandwidth or other limitations (e.g., power limitations, heat limitations).

Thus, after the ultrasound signals are digitized, the digitized signals may be compressed using compression techniques. In some embodiments, the signals are digitized using lossless compression methods. In some embodiments, lossless compression methods used may include using an H.261 codec, run length encoding (RLE), Lempel-Ziv-Welch (LZW), binary cluster (BL) universal code, frequency domain based lossless compression, Huffman coding, low complexity lossless compression for images (LOCO-I), or context-based adaptive lossless image codec (CALIC). In some embodiments, the signals are digitized using lossy compression methods. In some embodiments, lossy compression or encoding methods used may include encoding, such as joint photographic experts group (JPEG), motion picture experts group (MPEG), H.261, or using an inverse kinematics (IK) algorithm, discrete cosine transform (DCT), discrete wavelet transform (DWT), continuous wavelet transform (CWT), multi fractal compression, WavePDT compression, block-based motion compensation, log compression, or gamma compression.

The type (e.g., lossless or lossy) or degree of compression applied to the digitized signal may vary based on available bandwidth and imaging data size. This may allow the ultrasound imaging probe to minimize compression, thus increasing image quality and/or decreasing the delay between collection and presentation of the imaging data (e.g., via computing device 130).

In some embodiments, the type or degree of the compression applied to the digitized signal is based on whether the ultrasound imaging probe is collecting 2D, 3D, or 4D imaging data. In some embodiments, 2D imaging data may be small enough that it does not require compression prior to being sent to the computing device. On the other hand, 4D imaging data may be too large to send to the computing device without first being compressed (e.g., due to bandwidth limitations).

In some embodiments, the type or degree of the compression applied to the digitized signal is based on the organ(s) for which the ultrasound imaging probe is collecting imaging data. For example, if the organ(s) for which the ultrasound imaging probe is relatively simple (e.g., a liver), the digitized signal associated with the imaging data may not require compression. By comparison, digitized signal associated with imaging data for a complex organ(s) (e.g., a heart) may require compression before sending the data to the computing device.

In some embodiments, the type or degree of the compression applied to the digitized signal is based on the bandwidth of the connection (e.g., link 120) between the ultrasound imaging probe and the computing device. For example, if the ultrasound imaging probe is connected to the computing device via USB4, the digitized signal associated with the imaging data may not require much or any compression due to the relatively high bandwidth of USB4. However, if the ultrasound imaging probe is connected to the computing device via Bluetooth, the digitized signal associated with the imaging data may require more compression due to the lower bandwidth of Bluetooth.

In some embodiments, the type or degree of the compression applied to the digitized signal can be based on image quality considerations (e.g., computing device 130). For example, if the screen size or resolution of the computing device is relatively small (e.g., on a smartphone), the device may not be able to display a full-quality image based on the imaging data. Accordingly, the ultrasound imaging probe may apply a greater degree of compression without materially impacting the quality of the image displayed at or by the computing device. However, if the computing device's screen size is large or its resolution is high (e.g., on a computer monitor or a television), the ultrasound imaging probe may not compress or may apply a lossless compression to the digitized signal associated with the imaging data to avoid decreasing image quality.

In addition, or as an alternative to compression, the ultrasound imaging probe may apply beamforming to further account for bandwidth limitations. In some embodiments, the ultrasound imaging probe adds transducer signals together in the analog domain. For example, an ultrasound imaging probe with N transducers might arrange ultrasound data in M columns and in MN rows. This may reduce data rate by a factor of N/M. However, such a procedure may not work for 3D or 4D imaging, as information would be lost in one direction. It may be acceptable for 2D imaging, for instance, in conjunction with additional signal processing techniques, such as band limiting the data (by filtering out high frequency content) or utilizing other beamforming techniques which utilize FPGAs.

At any rate, beamforming may be applied to preserve as much information as possible while still compressing the ultrasound data to meet limitations (e.g., bandwidth limitations). In some embodiments, an ultrasound imaging probe having N transducer elements groups A elements together with appropriate beamforming (adding delay between samples before adding together). This may reduce the data rate by a factor of A by creating N/A micro beams, while still preserving spatial information. Moreover, the reduction can be followed by another reduction, for instance, using another factor of B, where the N/A beams are further delayed, weighed and summed to create A/(A*B) beams. In some embodiments, signal processing (e.g., analog or digital signal processing) computations are done in an integrated circuit (e.g., to decrease cost or power consumption).

In some embodiments, digitized signals from N transducers are weighted, delayed, and summed with B elements to create a microbeam. To reduce complexity, the ADC resolution and quantization of weights may be optimized to minimize cost. For example, ADCs typically used in high quality beam formation may be in the 12- to 14-bit range. In some embodiments, the ADC can be of lower resolution (e.g., 6-9 bits) to reduce size or cost. In some embodiments, the circuit needed to create all the beams can be implemented in an integrated circuit (e.g., to reduce power, cost, or size). Such an integrated circuit can have inputs from an external interface (e.g., an external interface of the computing device) to instruct the circuit on intended operation. The circuit can then implement the appropriate beamforming and date rate intended and direct data back to the computing device. The integrated circuit may create a multiplicity of outputs. In some embodiments, the outputs are combined into a high-speed data link suitable for an USB interface, such as a single or multiple serial link. In another implementation, the integrated circuit will feed P output lanes to another chip where a parallel to serial conversion is implemented.

In a fourth operation 340, the ultrasound imaging probe can convert the digital signal to a serial transmission. The ultrasound imaging probe may use a serializer to convert the signal. The serializer may comprise a plurality of electronic components, such as flip flops and multiplexers, to serialize the digital signal. In some embodiments, the ultrasound imaging probe serializes the digital signal using a dedicated integrated circuit (e.g., another dedicated integrated circuit inside the probe).

In some embodiments, the imaging data (e.g., the digitized signal, the serialized signal) is stored in a memory buffer (e.g., an elastic memory buffer). For example, a memory buffer may allow the portable ultrasound probe to temporarily store imaging data (e.g., after compression) during times when the amount of data to be sent to the computing device exceeds the available bandwidth, as discussed herein. The buffer may then send the imaging data stored therein during times when more bandwidth is available.

In a fifth operation 350, the ultrasound imaging probe provides the serial bit stream to an external computing device. The serial bit stream travels over a high-speed serial connection, such as USB4.

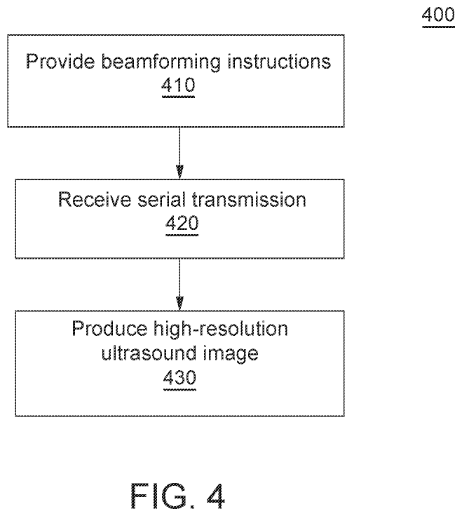

FIG. 4 illustrates a process 400 for producing an ultrasound image from a received bit stream. The bit stream may be received at a computing device. In some embodiments, the production of the ultrasound image may be performed on multiple computers. For example, some post-processing operations may be implemented on a first computer and other post-processing operations may be implemented on a second computer. Computing devices may transmit the digitized ultrasound signal to other computing devices for further processing via a network, via additional high-speed connections, or by using cloud computing.

In a first operation 410, a computing device provides beamforming instructions to a high-speed transmission interface of an ultrasound imaging probe via the high-speed transmission communication link. In other embodiments, the computing device may perform beamforming on received raw ultrasound data. For example, the beamforming instructions may comprise delays for digitized pulses of sine waves oscillating at an ultrasonic frequency. The ultrasound imaging probe may convert (e.g., via a DAC) the digital instructions to an analog signal before applying it to an array of ultrasonic transducer elements (e.g., pMUTs or cMUTs). The instructions may include assigning time delays to particular transducer elements, which may be implemented by programmable delay units communicatively coupled to the transducer elements. Pressure waves emitted by the transducer elements may constructively or destructively interfere, based on these delays, modifying the shape and direction of the output transducer wave. This transmitted wave is reflected by tissue (e.g., organ tissue) in the subject's body. By changing the values of the delays, via additional beamforming instructions, the computing device may cause the ultrasound imaging probe to scan an ultrasound beam in a pattern across the tissue. The physical properties (amplitude, phase, frequency, etc.) of the reflected wave are affected by properties (e.g., density) of the tissue from which they are reflected. The reflected wave signals are converted into electrical signals by the transducer elements, which apply preprocessing to correct errors associated with physical degradation of the reflected wave. Before or after preprocessing, the signal can be digitized and then converted into a serial bit stream by a serializer component.

In a second operation 420, the computing device receives a serial transmission over a high-speed serial connection from the ultrasound imaging device. The computing device receives the transmission via a high-throughput serial interface, such as an interface for USB4. For example, the data may be sent over a Thunderbolt 3 interface or a Thunderbolt 4 interface.

In a third operation 430, the computing device produces a high-resolution ultrasound image from the serial transmission. The computing device may implement one or more post-processing algorithms to generate the high-resolution image. Post-processing may enable an operator of the computing system to manipulate the image prior to display. Post-processing algorithms may include black/white inversion, freeze frame, frame averaging, or read zoom. Post-processing algorithms may also modify the digital image information sent over the high-speed interface. Such postprocessing algorithms may include thresholding, smoothing, contrast management, filtering, measurements and region-of-interest definition.

### Illustration of the Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An ultrasonic imaging system, comprising: an ultrasound imaging probe comprising an ultrasonic transducer and preprocessing circuitry, the ultrasonic transducer being configured to produce an electrical signal from an ultrasonic pressure wave and comprising a transducer element, the preprocessing circuitry being electrically coupled to the ultrasonic transducer and comprising a signal converter and a signal integrator, the signal converter being configured to condition a signal from the transducer element and convert the signal to a digital signal, the signal integrator being configured to combine the digital signal into a transmission signal with at least a 10 Gigabit per second (Gbps) data rate and transmit the transmission signal; a computing device configured to receive the transmission signal and implement a signal processing operation on the transmission signal to produce an ultrasound image; and a link for communicatively coupling the computing device and the signal integrator of the ultrasound imaging probe.

Clause 2. The ultrasonic imaging system of Clause 1, wherein the computing device is further configured to generate beamforming instructions.

Clause 3. The ultrasonic imaging system of Clause 2, wherein the computing device is further configured to direct the beamforming instructions to the ultrasound imaging probe via the link.

Clause 4. The ultrasonic imaging system of any of the preceding Clauses, wherein the ultrasonic transducer is a piezoelectric micromachined ultrasonic transducer (pMUT).

Clause 5. The ultrasonic imaging system of any of the preceding Clauses, wherein the ultrasonic transducer is a capacitive micromachined ultrasonic transducer (cMUT).

Clause 6. The ultrasonic imaging system of any of the preceding Clauses, wherein the ultrasonic transducer comprises a non-silicon-based piezo material.

Clause 7. The ultrasonic imaging system of Clause 6, wherein the non-silicon-based piezo material comprises aluminum nitride (AlN) or lead zirconate titanate (PZT).

Clause 8. The ultrasonic imaging system of any of the preceding Clauses, wherein the transmission signal has a transmission speed of up to 40 Gbps.

Clause 9. The ultrasonic imaging system of any of the preceding Clauses, wherein the transmission signal has a transmission speed of up to 80 Gbps.

Clause 10. The ultrasonic imaging system of any of the preceding Clauses, wherein the pre-processing circuitry performs one or more of gain compensation, selective enhancement, log compression, fill-in interpolation, edge enhancement, image updating, or write zoom.

Clause 11. The ultrasonic imaging system of any of the preceding Clauses, wherein the ultrasonic transducer comprises a plurality of transducer elements.

Clause 12. The ultrasonic imaging system of Clause 11, wherein the plurality of transducer elements is configured in an array.

Clause 13. The ultrasonic imaging system of Clause 12, wherein the array has between 2 and 100,000 transducer elements.

Clause 14. The ultrasonic imaging system of any of the preceding Clauses, wherein the preprocessing circuitry comprises between 1 and 1024 ultrasonic transducer channels.

Clause 15. The ultrasonic imaging system of any of the preceding Clauses, wherein the preprocessing circuitry further comprises a low noise amplifier or an analog-to-digital converter (ADC).

Clause 16. The ultrasonic imaging system of Clause 15, wherein the ADC provides a resolution between 8 and 16 bits.

Clause 17. The ultrasonic imaging system of Clause 15, wherein the ADC provides a resolution between 1 and 8 bits.

Clause 18. The ultrasonic imaging system of Clause 15, wherein the ADC operates at a frequency above 10 MHz.

Clause 19. The ultrasonic imaging system of any of the preceding Clauses, wherein the computing device comprises a desktop, a laptop, a personal digital assistant, a tablet computer, or a smartphone.

Clause 20. The ultrasonic imaging system of any of the preceding Clauses, wherein the computing device comprises a smartphone that uses an Android or iOS operating system.

Clause 21. The ultrasonic imaging system of any of the preceding Clauses, wherein the ultrasound image comprises a 3D image or a 4D image.

Clause 22. The ultrasonic imaging system of Clause 21, wherein the computing device is further configured to implement a signal processing operation on the transmission signal to produce other ultrasound images.

Clause 23. The ultrasonic imaging system of Clause 22, wherein the other ultrasound images have framerates of up to 1000 frames per second (FPS).

Clause 24. The ultrasonic imaging system of any of the preceding Clauses, wherein the link supplies power from the computing device to the ultrasound imaging probe.

Clause 25. The ultrasonic imaging system of any of the preceding Clauses, wherein the signal converter implements microbeamforming on the signal from the transducer element.

Clause 26. The ultrasonic imaging system of any of the preceding Clauses, wherein the computing device is further configured to provide the transmission signal or the ultrasound image to a deep learning module trained using ultrasound imaging data and configured to produce beamforming instructions when provided with ultrasound imaging data.

Clause 27. The ultrasonic imaging system of any of the preceding Clauses, wherein the ultrasonic transducer further comprises an array of transducer elements that includes the transducer element, the preprocessing circuitry further comprises LNAs, and each respective transducer element in the array of transducer elements is connected to a respective LNA configured to amplify a respective signal from the respective transducer element.

Clause 28. The ultrasonic imaging system of any of the preceding Clauses, wherein the ultrasonic transducer further comprises an array of transducer elements that includes the transducer element, the signal converter of the preprocessing circuitry comprises ADCs, and each respective transducer element in the array of transducer elements is connected to a respective ADC configured to condition a respective signal from the respective transducer element.

Clause 29. The ultrasonic imaging system of any of the preceding Clauses, wherein the link defines a bandwidth limitation, and the preprocessing circuitry is configured to apply a compression to the transmission signal prior to transmitting the transmission signal based on a determination that a size of the transmission signal will exceed the bandwidth limitation.

Clause 30. The ultrasonic imaging system of Clause 29, wherein a type of the compression applied to the transmission signal is based on an amount by which the size of the transmission signal will exceed the bandwidth limitation.

Clause 31. The ultrasonic imaging system of any of the preceding Clauses, wherein the link defines a bandwidth limitation, and the preprocessing circuitry is configured to forgo applying a compression to the transmission signal prior to transmitting the transmission signal based on a determination that a size of the transmission signal will not exceed the bandwidth limitation.

Clause 32. The ultrasonic imaging system of any of the preceding Clauses, wherein the link defines a bandwidth limitation, and the ultrasound imaging probe further comprises a buffer configured to store a portion of the transmission signal while a size of the transmission signal exceeds the bandwidth limitation.

Clause 33. The ultrasonic imaging system of any of the preceding Clauses, wherein the link comprises a single serial link.

Clause 34. The ultrasonic imaging system of any of the preceding Clauses, wherein the link comprises multiple serial links.

Clause 35. The ultrasonic imaging system of any of the preceding Clauses, wherein the link comprises a USB4 link, a USB3 link, a PCI-E link, or a PXIE link.

Clause 36. The ultrasonic imaging system of any of the preceding Clauses, wherein the link comprises a wireless connection.

Clause 37. A method for producing an ultrasound image, comprising: receiving ultrasonic signals; receiving beamforming instructions via a high-speed serial link; pre-processing the ultrasonic signals responsive to the beamforming instructions by producing a bit stream with a data rate of at least 10 Gigabit per second (Gbps) from the ultrasonic signals; and transmitting the bit stream through the high-speed serial link.

Clause 38. The method of Clause 37, wherein the bit stream is transmitted with a transmission speed of up to 40 Gbps.

Clause 39. The method of any of Clauses 37 to 38, wherein the bit stream is transmitted with a transmission speed of up to 80 Gbps.

Clause 40. The method of any of Clauses 37 to 39, wherein pre-processing the ultrasonic signals comprises applying one or more of gain compensation, selective enhancement, log compression, fill-in interpolation, edge enhancement, image updating, or write zoom.

Clause 41. The method of any of Clauses 37 to 40, wherein pre-processing the ultrasonic signals comprises applying low noise amplification or analog-to-digital conversion.

Clause 42. The method of Clause 39, wherein pre-processing the ultrasonic signals provides a resolution between 8 and 16 bits.

Clause 43. The method of Clause 39, wherein pre-processing the ultrasonic signals provides a resolution between 1 and 8 bits.

Clause 44. The method of any of Clauses 37 to 43, wherein the ultrasound image comprises a 3D image or a 4D image.

Clause 45. The method of any of Clauses 37 to 44, further comprising producing an ultrasound image based on the bitstream.

Clause 46. The method of any of Clauses 37 to 45, wherein the ultrasound image has a framerate of up to 1000 frames per second (FPS).

Clause 47. The method of any of Clauses 37 to 46, wherein pre-processing the ultrasonic signals comprises microbeamforming on a subset of the ultrasonic signals.

Clause 48. A method for producing one or more ultrasound images, comprising: providing, via a high-speed communication link, a plurality of beamforming instructions to an ultrasound imaging probe, wherein the high-speed communication link is configured to provide a data rate of at least 10 Gbps; receiving, from the ultrasound imaging probe and via the high-speed communication link, a serial bit stream comprising a digitized ultrasound signal; and producing an ultrasound image from the digitized ultrasound signal.

Clause 49. The method of Clause 48, wherein the serial bit stream is transmitted with a transmission speed of up to 40 Gbps.

Clause 50. The method of any of Clauses 48 to 49, wherein the serial bit stream is transmitted with a transmission speed of up to 80 Gbps.

Clause 51. The method of any of Clauses 48 to 50, wherein the ultrasound image is a 3D image or a 4D image.

Clause 52. The method of any of Clauses 48 to 51, wherein the one or more ultrasound images is a plurality of ultrasound images.

Clause 53. The method of any of Clauses 48 to 52, wherein the one or more ultrasound images have framerates of up to 1000 frames per second (FPS).

Clause 54. The method of any of Clauses 48 to 53, wherein the plurality of beamforming instructions comprises delays for digitized pulses of sine waves oscillating at an ultrasonic frequency.

Clause 55. The method of Clause 54, wherein delays are assigned to particular transducer elements.

Clause 56. The method of any of Clauses 48 to 55, wherein producing the ultrasound image from the digitized ultrasound signal comprises performing a post-processing algorithm on the digitized ultrasound signal.

Clause 57. The method of Clause 56, wherein the post-processing algorithm comprises thresholding, smoothing, contrast management, filtering, measurements, and region-of-interest definition.

Clause 58. An ultrasonic probe comprising any of the features or components of any of the preceding Clauses.

Clause 59. An ultrasonic imaging system comprising any of the features or components of any of the preceding Clauses.

Clause 60. A method of any of the preceding Clauses, utilizing any of the steps, features, or components of any of the preceding Clauses.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

What is claimed is:

1. An ultrasonic imaging system, comprising:
an ultrasound imaging probe comprising an ultrasonic transducer and preprocessing circuitry, the ultrasonic transducer being configured to produce an electrical signal from an ultrasonic pressure wave and comprising a transducer element, the preprocessing circuitry being electrically coupled to the ultrasonic transducer and comprising a signal converter and a signal integrator, the signal converter being configured to condition a signal from the transducer element and convert the signal to a digital signal, the signal integrator being configured to combine the digital signal into a transmission signal with at least a 10 Gigabit per second (Gbps) data rate and transmit the transmission signal;
a computing device configured to receive the transmission signal and implement a signal processing operation on the transmission signal to produce an ultrasound image; and
a link for communicatively coupling the computing device and the signal integrator of the ultrasound imaging probe.

2. The ultrasonic imaging system of claim 1, wherein the ultrasonic transducer is a piezoelectric micromachined ultrasonic transducer (pMUT).

3. The ultrasonic imaging system of claim 1, wherein the ultrasonic transducer is a capacitive micromachined ultrasonic transducer (cMUT).

4. The ultrasonic imaging system of claim 1, wherein the transmission signal has a transmission speed of up to 40 Gbps.

5. The ultrasonic imaging system of claim 1, wherein the preprocessing circuitry performs one or more of gain compensation, selective enhancement, log compression, fill-in interpolation, edge enhancement, image updating, or write zoom.

6. The ultrasonic imaging system of claim 1, wherein the ultrasound image is a 3D image or a 4D image.

7. The ultrasonic imaging system of claim 6, wherein the computing device is further configured to implement a signal processing operation on the transmission signal to produce other ultrasound images.

8. The ultrasonic imaging system of claim 1, wherein the link supplies power from the computing device to the ultrasound imaging probe.

9. The ultrasonic imaging system of claim 1, wherein the signal converter implements microbeamforming on the signal from the transducer element.

10. The ultrasonic imaging system of claim 1, wherein the computing device is further configured to provide the transmission signal or the ultrasound image to a deep learning module trained using ultrasound imaging data and configured to produce beamforming instructions when provided with ultrasound imaging data.

11. The ultrasonic imaging system of claim 1, wherein the ultrasonic transducer further comprises an array of transducer elements that includes the transducer element, the preprocessing circuitry further comprises LNAs, and each respective transducer element in the array of transducer elements is connected to a respective LNA configured to amplify a respective signal from the respective transducer element.

12. The ultrasonic imaging system of claim 1, wherein the ultrasonic transducer further comprises an array of transducer elements that includes the transducer element, the signal converter of the preprocessing circuitry comprises ADCs, and each respective transducer element in the array of transducer elements is connected to a respective ADC configured to condition a respective signal from the respective transducer element.

13. The ultrasonic imaging system of claim 1, wherein the link defines a bandwidth limitation, and the preprocessing circuitry is configured to apply a compression to the transmission signal prior to transmitting the transmission signal based on a determination that a size of the transmission signal will exceed the bandwidth limitation.

14. The ultrasonic imaging system of claim 13, wherein a type of the compression applied to the transmission signal is based on an amount by which the size of the transmission signal will exceed the bandwidth limitation.

15. The ultrasonic imaging system of claim 1, wherein the link comprises a single serial link.

16. The ultrasonic imaging system of claim 1, wherein the link comprises multiple serial links.

17. The ultrasonic imaging system of claim 1, wherein the link comprises a USB4 link, a USB3 link, a PCI-E link, or a PXIE link.

18. A method for producing an ultrasound image, comprising:
receiving ultrasonic signals;
receiving beamforming instructions via a high-speed serial link;
pre-processing the ultrasonic signals responsive to the beamforming instructions by producing a bit stream with a data rate of at least 10 Gigabit per second (Gbps) from the ultrasonic signals;
transmitting the bit stream through the high-speed serial link.

19. The method of claim 18, wherein the bit stream is transmitted with a transmission speed of up to 40 Gbps.

20. The method of claim 18, wherein pre-processing the ultrasonic signals comprises applying one or more of gain compensation, selective enhancement, log compression, fill-in interpolation, edge enhancement, image updating, or write zoom.

21. The method of claim 18, wherein the ultrasound image comprises a 3D image or a 4D image.

22. The method of claim 18, wherein pre-processing the ultrasonic signals comprises microbeamforming on a subset of the ultrasonic signals.
